SEQUENCE LISTING (4 sequences)

SEQ ID NO: 1 - DNA, 20 bp, Artificial Sequence
  Sequence of upstream primer for hTERT
cggaagagtgtctggagcaa

SEQ ID NO: 2 - DNA, 19 bp, Artificial Sequence
  Sequence of downstream primer for hTERT
ggatgaagcggagtctgga

SEQ ID NO: 3 - DNA, 21 bp, Artificial Sequence
  Sequence of upstream primer for AFP
ccagaaactagtcctggatgt

SEQ ID NO: 4 - DNA, 20 bp, Artificial Sequence
  Sequence of downstream primer for AFP
cgtggtcagtttgcagcatt

(19) United States
(12) Patent Application Publication
(10) Pub. No.: US 2007/0178461 A1
(43) Pub. Date: Aug. 2, 2007

(54) Cancer diagnostic method

(76) Inventors: Norimasa Miura, Yonago-shi (JP); Goshi Shiota, Yonago-shi (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/580,415

(22) PCT Filed: Nov. 18, 2004
(86) PCT No.: PCT/JP04/17542

§ 371 Date: Apr. 3, 2007

(30) Foreign Application Priority Data
Nov. 21, 2003 (JP) 2003-392875

(51) Int. Cl. C12Q 1/68; C12P 19/34

(52) U.S. Cl. 435/6; 435/91.2

(57) ABSTRACT

To provide a cancer diagnostic method capable of detecting evidence presenting the presence of cancer cells in early stage cancer. Cancer diagnostic method comprised of; a process to obtain the sample containing RNA only as a somatic cell and cancer cell fraction from body fluid and a process having a reverse transcription reaction step to generate cDNA using reverse transcriptase from the sample containing said RNA only and a PCR reaction step utilizing fluorescent dye using the following primers for hTERT, CGGAAGAGTGTCTGGAGCAA and GGATGAAGCGGAGTCTGGA to quantify the PCR product amplified by the PCR reaction using the fluorescent dye binding to the PCR product.

### FIELD OF THE INVENTION

The present invention relates to a method for detecting cancer, and more specifically for a cancer diagnostic method to identify the presence or absence of tumor cells in the early stage of cancer.

### BACKGROUND OF THE INVENTION

Approximately 300,000 cancer cases are newly diagnosed every year in Japan.

In addition, one every four or five patients die from cancer or complications associated with cancer therapy in Japan. Therefore, enormous efforts have been devoted to improve therapies for cancer or complications associated with cancer therapy.

Furthermore, due to the fact that the early diagnosis of cancer tends to result in a high cure rate, the improvement and the development of a diagnostic method to detect early stage of cancer with high accuracy has been underway.

In other words, most of cancer patients do not die from cancer or complicated tumors associated with cancer therapy. Cancer patients are rather seriously affected by a number of tumor colonies formed by malignant cells dissociated from primary tumor mass and metastasized to the other area different from the site where the primary tumor cells exist.

This suggests that it is highly likely to be able to overcome cancer or extend to a patient's life by identifying and detecting early stage cancer in patients to reduce its size or remove such early stage cancer by means of medical excision, medical anticancer therapy, surgery, radiation therapy and chemotherapy with anticancer drugs and the combination thereof.

However, the identification or detection of cancer in patients is a difficult task, especially for the detection of a metastatic colony, which is a sign of malignant cancer, not to mention the removal thereof, which makes cancer therapy clinically challenging.

Metastasis of cancer includes the following complex processes.
1) Expansion of cancer cells from the primary site to neighboring tissues
2) Penetration of cancer cells into lumens and blood vessels.
3) Transfer and release of cancer cells to distant sites via the circulatory system
4) Re-invasion of cancer cells into tissues to be localized
5) Survival of cancer cells at the new site and adaptation to a new environment to form blood vessels for tumor proliferation

More specifically, it is assumed that cancer cells invade neighboring tissues at an early stage to destroy tissue barriers, and the cancer cells invade tissue spaces and capillary blood vessels at an early developmental stage as a solid tumor (namely the stage when the tumor contains between 10⁴ to 10⁶ tumor cells). At this time, most of the cancer cells are eliminated via apoptotic process or immuno-competent cells, killed by immuno-competent cells, resulting in cell death, or they become dormant. This is due to the fact that cancer cells at this stage cannot survive or grow in an ectopic environment.

In the present invention, there is no detection method invented to detect such a small tumor at an early stage.

The most developed method currently available is a highly sensitive method to be used for the diagnosis of specific types of tumors when a tumor reaches a certain size.

For such diagnostic method, for example, mammography, which is capable of detecting 2×10⁸ mammary tumor cells in breasts, has been developed.

In the case of breast cancer, most of the cancer cells released at an early stage are believed to die. However, when the number of cancer cells becomes 10⁶ to 10⁹, cancer cell clones becomes genetically unstable to further mutate at the gene level to generate highly proliferative and aggressive mutant cells for several generations. Such mutant cells are highly likely to survive as secondary tumors.

As described above, the detection of tumor at an early stage is not possible, and for example, the diagnosis of most types of cancer such as, pancreas, stomach, ovary, kidney, lung and liver cancers are performed only when the number of the cancer cells reaches 10¹⁰ to 10¹², namely at a highly advanced stage. At this stage, the tumor may have already invaded neighboring tissues and metastasized.

Conventionally, a diagnostic examination has been developed to monitor the metastasis of cancer or the effectiveness of cancer therapy when a treatment plan is established which considers the difficulty of the radical treatment of cancer, the complexity of tumor metastasis, the side effects of drugs used in chemotherapy and the stress and anxiety of the patient against the treatment.

As a result of efforts over the last 20 years, various diagnostic examination methods have been developed and their usefulness recognized.

More specifically, such first approach was an immunoassay formulation of carcinoembryonic antigen (CEA), which is considered to be an embryonic antigen produced in the digestive organs of the fetus, expressed on specific cancer cells such as digestive organ cancers including the colon/rectum, pancreas and biliary tract cancers and lung cancer.

With the advent of new techniques such as immunochemical method such as radio immuno assay (RIA) and enzyme immunoassay (Enzyme-Linked Immuno-sorbent Assay (EIA (or ELISA)), a number of diagnostic methods have developed including RIA (beads solid phase method) using CEA as a tumor marker and reverse passive haemagglutination technique (R-PHA) and RIA (beads solid phase method) using α-fetoprotein (AFP) as a tumor marker, enzyme immunoassay (EIA) and RIA (beads solid phase method) using prostate specific-antigen as a tumor marker, RIA (beads solid phase method) using CA15-3 as a tumor marker, enzyme immunoassay (EIA) using CA50 as a marker, RIA (beads solid phase method) using CA125 as a marker and enzyme immunoassay (EIA) using PIVKA II ([protein induced by vitamin K absence or antagonist]II).

However, it has been understood that such diagnostic methods are not efficient for the detection of some antigens such as CEA, AFP, CA15-3, CA50, CA125 and PIVKA II since these antigens are not normally expected to be present in serum, and when their presence is detected, cancer in a patient is already in a quite advanced stage and there is almost no hope for the patient to survive.

However, in the last decade, one promising tumor marker has been considered to be useful for the early detection of cancer and various efforts in research and development for its clinical application have been continuously carried out.

This tumor marker is called “telomerase (hTERT).”

Telomerase (hTERT) is a malignant tumor specific antigen (enzyme) produced and expressed in 90% of tumors. Since the discovery of its activity in 1994 (See Kim N W.Science.23;266 : 2011-2015(1994)), the discovery of gene and its functional analyses have been carried out. However, despite of its specificity, telomerase (hTERT) was clinically found only in an excised tissue in which a tumor has already been formed and metastasized and it was impossible to easily detect telomerase (hTERT) in the blood, unlike current clinical examinations. In addition, even telomerase (hTERT) is detected, the accurate quantitative detection of telomerase has been a problem as it cannot completely eliminate the possibility of the contamination of other cells (such as lymphocytes) that produce a trace amount of telomerase (hTERT).

In 2000, although it has been reported hTERT can be quantitatively detected in blood samples from breast cancer patients (Chen X Q. Clin Cancer Res.6: 3823-3826(2000)), its sensitivity was less than 60% and cannot be used for clinical applications.

High sensitivity and reliable quantification are necessary factors as an effective diagnostic examination.

Furthermore, the development of a blood test capable of detecting the presence of a single cancer cell in 1 ml of blood, equivalent to circulating 3000-4000 tumor cells in total, is useful. An inoculation study to test the deposition of cancer cells in animals indicates that such a number of cell is actually enough to deposit cancer. Furthermore, when such 3000-4000 circulating cells account for 0.01% of a total tumor cells in the body, it means there are as many as 4×10⁷ tumor cells exist, and none of the currently available method is capable of detecting a tumor having such a number of cells.

Therefore, when tumor cells are released at an early stage of cancer, cancer can be detected by the development of a method having such sensitivity. In addition, quantitative method is useful to assess the cancer load if tumor cells are released proportional to the tumor size.

Furthermore, when various DNA, protein and RNA are released from cancer cells and immuno-competent cells into the blood as a result of battles between the cancer cells and immuno-competent cells, it is possible that RNA can be detected. In such a case, the detection of cancer specific RNA indicates the earliest event during metastasis. However, there has been no knowledge of the presence of circulating cancer cells in the very early stages of cancer.

For the above reasons, the development of a method to fix circulating cells with metastatic ability prior to the establishment of secondary tumor, especially to identify such cells in early stage cancer is desired. The development of a method to detect evidence indicating the presence of cancer cells in the blood, to recognize the expression level in normal cells or to quite sensitively detect RNA derived from cancer cells at 1-10 copy level results in providing clinically useful information.

Thanks to the improvement of various assay systems, the quantification of tumor tissue has become possible. The development of such a highly sensitive quantification method is now plausible and the quantification of RNA derived from various cancer cell types are believed to be technically ready to be achieved.

The present invention has been achieved in view of the above problems, and it is therefore an object of the present invention to provide a cancer diagnostic method to detect the presence of cancer cells in the blood in the early stages of cancer.

### DISCLOSURE OF THE INVENTION

The cancer diagnostic method described in claim 1 is comprised of;

a process to obtain the sample containing RNA only as a somatic cell and cancer cell fraction from body fluid and

a process having a reverse transcription reaction step to generate cDNA using reverse transcriptase from the sample containing RNA and a PCR reaction step utilizing fluorescent dye using the following primers for hTERT, CGGAAGAGTGTCTGGAGCAA and GGATGAAGCGGAGTCTGGA to quantify the PCR product amplified by the PCR reaction using fluorescent dye binding to the PCR product.

In addition, “body fluid,” a term used in the present specification, indicates body fluid including blood, lymph fluid and the like.

The cancer diagnostic method described in claim 2 is comprised of;

a process to obtain the sample containing only RNA as a somatic cell and cancer cell component from body fluid,

a process having a reverse transcription reaction step to generate cDNA using reverse transcriptase from the sample containing RNA and a PCR reaction step utilizing fluorescent dye using the following primers for AFP, CCAGAAACTAGTCCTGGATGT and CGTGGTCAGTTTGCAGCATT to quantify the PCR product amplified by the PCR reaction using the fluorescent dye binding to the PCR product.

The cancer diagnostic method according to the present invention is capable of detecting the evidence indicating the presence of the cancer cells in the blood in the early stage cancer to eradicate the cancer cells at an early stage with medial approaches.

In addition, the presence or absence of cancer cells is accurately detected in comparison with a case in which telomerase (hTERT) or AFP is detected from the biopsy of established cancer tissue or metastasized tissue since the sample containing mRNA is obtained from the blood [in the present method].

In addition, by appropriately designing the primers used for the PCR reaction, the evidence indicating the presence of cancer cells in early stage cancer can be detected in the blood.

### DETAILED DESCRIPTION OF PREFERRED DRAWINGS

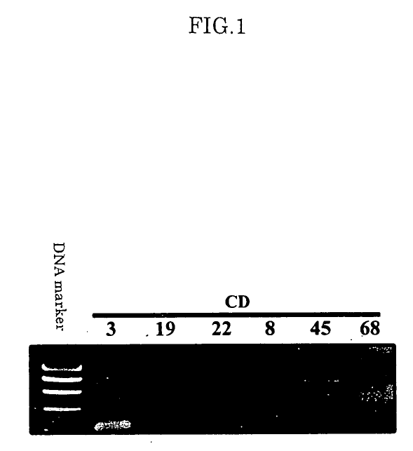

FIG. 1 shows the results of the PCR reaction confirming the removal of hematocytes since only T lymphocyte fractions (CD3, CD8; The PCR product amplified by PCR reaction was measured quantitatively using a fluorescent dye binding to the PCR product) were detected in the RNA extracted with the present method.

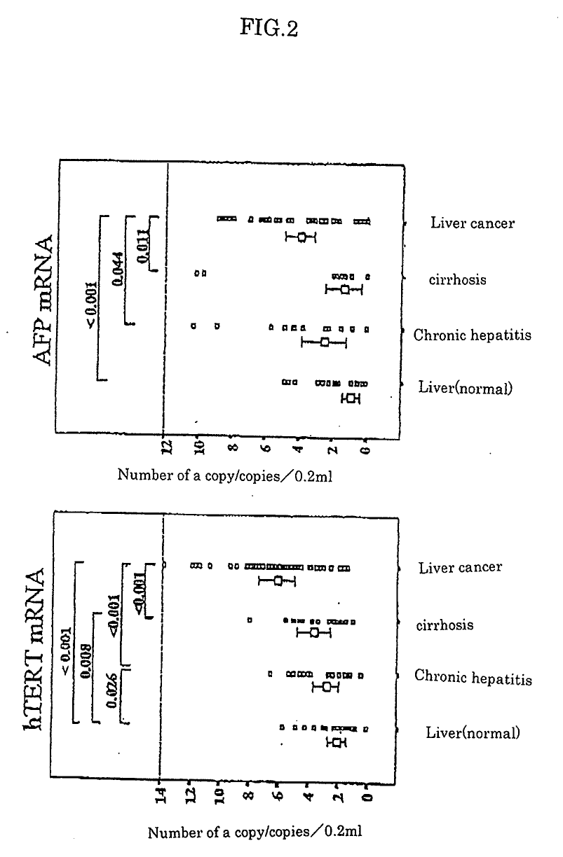

FIG. 2 shows a copy number indicating that the stepwise up-regulation of the telomerase and AFP gene expressions as the chronic hepatic diseases (hepatitis and liver cirrhosis) develop to liver cancer and statistically significant differences in each group are shown in number in the upper part of the figure. The 95% confidence interval is shown as an error bar on the left of the scatter diagram and the boxes between the error bars indicate mean values.

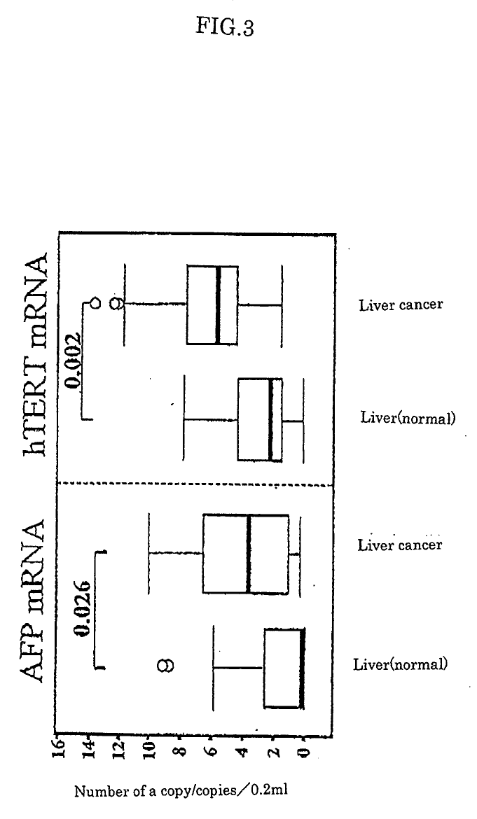

FIG. 3 is a box plot indicating that the cancer diagnostic method examining the gene expression (hTERT mRNA) according to the present invention is more effective than conventional tumor markers by using the statistically significant difference between liver cancer patients and normal individuals.

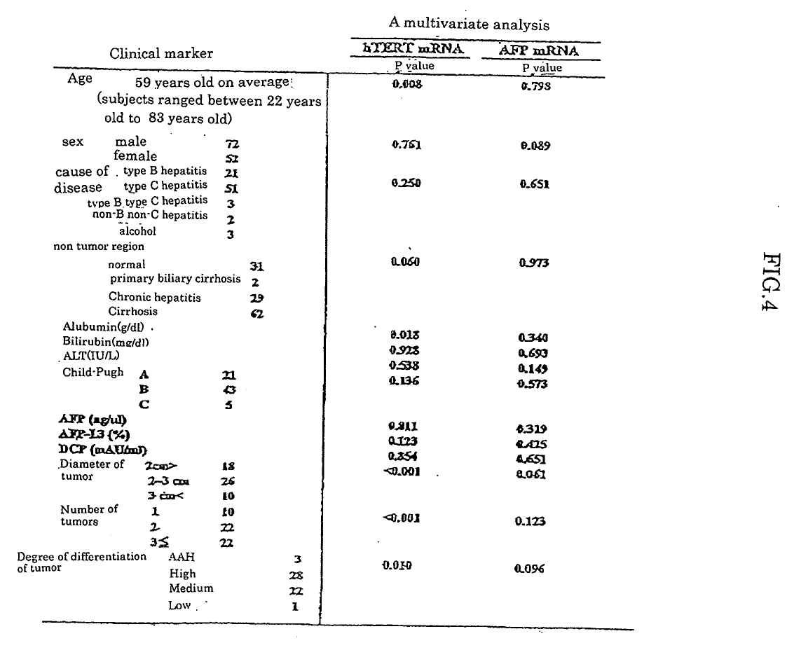

FIG. 4 is a result of multivariate analysis of comparison of various types of clinical examination results and findings by the quantified two gene expressions (hTERT mRNA and AFP mRNA).

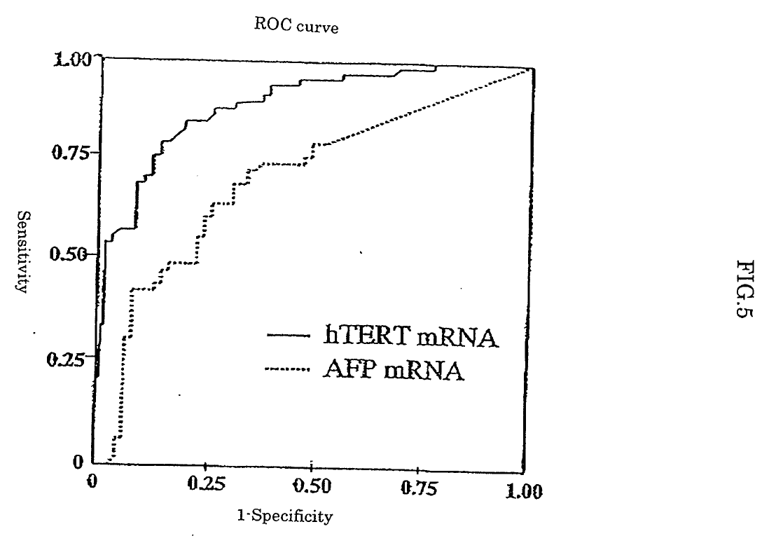

FIG. 5 is an ROC curve indicating sensitivity and specificity of quantification by the cancer diagnostic method of the two gene expressions (hTERT mRNA and AFP mRNA). (The ROC curve means a receiver operator characteristic curve analysis.)

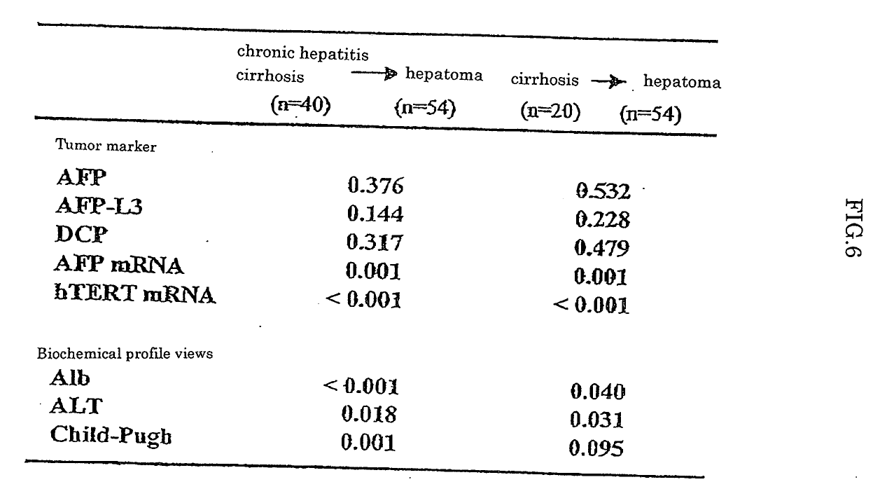

FIG. 6 is the result of multivariate analysis of correlation of the various clinical examination results and findings between the quantified value of the cancer diagnostic method using conventional tumor markers (AFP, AFP-L3, DCP) and the quantified values of the cancer diagnostic method by the two gene expressions (hTERT mRNA and AFP mRNA) during progression from chronic liver diseases to liver cancer.

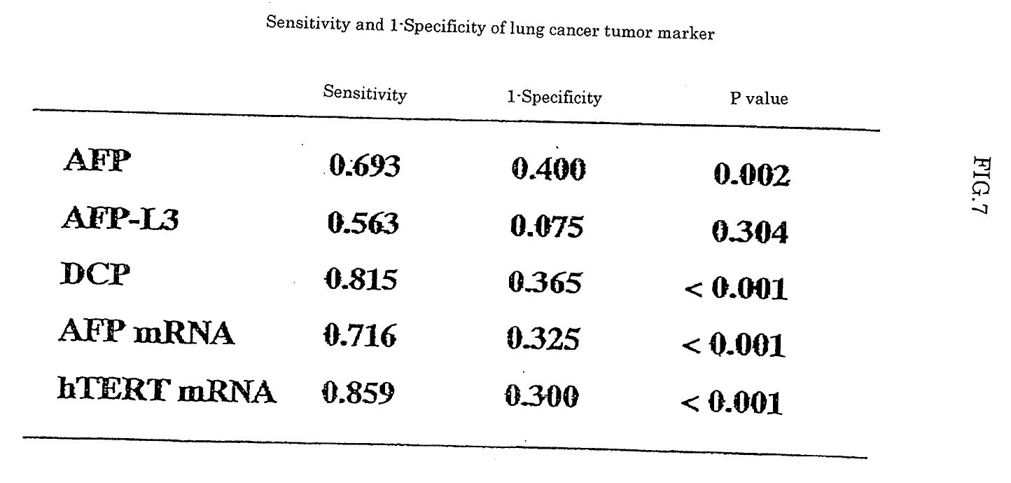

FIG. 7 is a figure comparing the sensitivity and specificity between the conventional tumor markers (AFP, AFP-L3, DCP) in liver cancer and the two gene expressions (hTERT mRNA and AFP mRNA) used in the present method during the progression from chronic liver diseases to liver cancer.

### BEST MODE FOR CARRYING OUT THE INVENTION

An example of the cancer diagnostic method according to the present invention is explained in detail hereafter.

In the cancer diagnostic method according to the present invention, first, a blood sample is collected from a subject (patient).

Then, a specimen containing RNA is extracted from the blood.

In this step, RNA, which is circulating in blood, has to be selectively extracted without being influenced as much as possible by other blood cells.

In order to achieve this purpose, the blood sample has to be processed promptly in the following manner once the body fluids are collected from the subject (patient).

1) When a blood collection tube is EDTA-free:

Body fluids (approximately 1-2 ml) collected under the consent obtained from a patient were centrifuged at 700-800 xg for 10 min at 4° C. The supernatant was transferred to a new RNase-free tube to further centrifuge at 1500 xg for 10 min at 4° C. Then, the supernatant was transferred to another RNase-free tube to further centrifuge at 1600-3000 xg for 10 min at 4° C. The sample should be stored at −80 ° C. for later use unless immediately used as a sample containing RNA.

2) When a blood collection tube is not EDTA-free:

The body fluid obtained in a similar manner to the above step 1) was centrifuged at 1500-1600 xg for 10 min at 40° C. The supernatant was transferred to a new RNase-free tube to further centrifuge at 1500 xg for 10 min at 4° C. Then, the supernatant was sterilized with a 0.22 μm filter. The sample should be stored at −80° C. for later use unless immediately used as a sample containing RNA.

Next, PCR reaction of the sample containing RNA was performed using the following primers for hTERT analysis: CGGAAGAGTGTCTGGAGCAA and GGATGAAGCGGAGTCTGGA. The amplified PCR product was quantitatively measured using a fluorescent dye binding to the PCR product. For AFP analysis, the following primers, CCAGAAACTAGTCCTGGATGT and CGTGGTCAGTTTGCAGCATT are used to perform PCR reaction, and then the amplified PCR product was quantitatively measured using a fluorescent dye binding to the PCR product.

More specifically, 175 μl of dilution buffer, lysis buffer (SV total RNA isolation system) or TRIzol reagent were used for each 100 μl of the sample containing RNA prepared in the above method 1) or 2) to extract RNA after DNase treatment according to an instruction manual (an instruction manual from SV total RNA isolation system in this case).

Twenty μl of RNA in 200 μl of RNase free water obtained through two elution processes or 1 μl of RNA in 10 μl of volume-adjusted solvent was used for RT-PCR reaction.

Next, in order to quantify in a single step, both a reverse transcription reaction to generate cDNA from RNA using reverse transcriptase and a quantitative PCR method using fluorescent dye (SYBR Green 1 (Roche) in this example) are carried out in a single tube.

In summary, the reaction mixture is prepared by adding 1-2 μl of fluorescent dye (SYBR Green 1, Roche in this case) for each 25 μl of the total volume of the components according to an instruction manual (One Step RT-PCR kit protocol (QIAGEN)), the expression level of RNA in the original sample is measured using an quantitative PCR machine (Light Cycler, Roche in this case) by following the instruction manual (One Step RT-PCR kit protocol (QIAGEN)).

At this time, the reaction is carried out 1) at 50° C. for 30 min as a reverse transcription reaction step, 2) at 95° C. for 15 min as an activation step, 3) 55 cycles of 3-step PCR reaction. The annealing temperature varies depending on the primers to be used. For example, CGGAAGAGTGTCTGGAGCAA and GGATGAAGCGGAGTCTGGA are used as hTERT primers and CCAGAAACTAGTCCTGGATGT and CGTGGTCAGTTTGCAGCATT are used as AFP primers. The data obtained will be compared and analyzed with the optimum cutoff values (some types of cancers use a plurality of cutoff values of markers to increase its specificity) statistically processed for each type of tumor to determine the presence or absence of cancer cells in the patient blood.

Next, the results will be explained.

FIG. 1 shows the results of the PCR reaction confirming the removal of hematocytes since only T lymphocyte fractions (CD3, CD8; The PCR product amplified by PCR reaction was measured quantitatively using a fluorescent dye binding to the PCR product) were detected in the RNA extracted using the cancer diagnostic method according to the present invention.

As clearly indicated in FIG. 1, the inventor(s) of the present invention confirmed the elimination of hematocytes, which is an important step for the diagnostic method according to the present invention, by checking mRNA of various hematocyte markers such as CD3, CD8, CD19, CD22, CD45 and CD68.

FIG. 2 shows a copy number indicating that the stepwise up-regulation of the telomerase and AFP gene expressions as hepatic diseases (hepatitis and liver cirrhosis) develop to liver cancer and statistically significant differences in each group are shown in number in the upper part of the figure. The 95% confidence interval is shown as an error bar on the left of the scatter diagram and the boxes between the error bars indicate mean values.

FIG. 3 is a box plot indicating that the cancer diagnostic method according to the present invention is more effective than conventional tumor markers by using the statistically significant difference between liver cancer patients and normal individuals.

FIGS. 2 and 3 indicate that the expression of hTERT mRNA is increased in a stepwise fashion during the progression and exacerbations of liver legion in the cancer diagnostic method according to the present invention.

FIG. 4 is a result of multivariate analysis of comparison of various types of clinical examination results and findings by the quantified two gene expressions (hTERT mRNA and AFP mRNA).

FIG. 4 indicates that the expression of hTERT mRNA is strongly correlated to tumor size, the number of tumors and differentiation degrees especially in terms of the development of cancer based on the multivariate analysis of comparison of the clinical examination (biochemical examination of liver function and serological examination such as the number of virus) and the clinical findings (tumor size, number of tumors and differentiation degrees). FIG. 5 is an ROC curve indicating sensitivity and specificity of quantification by the cancer diagnostic method of the two gene expressions (hTERT mRNA and AFP mRNA). (The ROC curve means a receiver operator characteristic curve analysis.)

As is clearly indicated in FIG. 5, the sensitivity and specificity of liver cancer, whose death rate is the fourth largest death rate in cancer, by the cancer diagnostic method using the gene expression (hTERT mRNA) according to the present invention were 88.2% and 68.7%, respectively.

On the other hand, the sensitivity and specificity by the cancer diagnostic method using the gene expression (AFP mRNA) were 70.1% and 65.8%, respectively.

In addition, the sensitivity and specificity of the cancer diagnostic method of gene expression (hTERT mRNA) according to the present invention during the development of liver cancer (For most of the liver carcinogenesis, the development of cancer includes virus chronic liver disease and when [the data] were statistically processed after eliminating normal individuals) are 85.9% and 70.0%, respectively, which are not less than the other tumor markers by any means.

FIG. 6 is a result of multivariate analysis of correlation of the various clinical examination results and findings between the quantified value of the cancer diagnostic method using conventional tumor markers (AFP, AFP-L3, DCP) and the quantified values of the cancer diagnostic method by the two gene expressions (hTERT mRNA and AFP mRNA) during the progression from chronic liver diseases to liver cancer.

FIG. 7 is a figure comparing the sensitivity and specificity between the cancer diagnostic method by the conventional tumor markers (AFP, AFP-L3, DCP) in liver cancer and the cancer diagnostic method using the two gene expressions (hTERT mRNA and AFP mRNA) during the progression from chronic liver diseases to liver cancer.

FIGS. 6 and 7 shows that FIGS. 6 and 7 show that when the expression of α-fetoprotein (AFP), the most reliable liver cancer marker, was compared using the cancer diagnostic method by the two gene expression (hTERT mRNA and AFP mRNA), the gene expression by the present invention (hTERT mRNA) had better sensitivity (69.3% for AFP, 85.9% for hTERT) and specificity (60.0% for AFP, 70.0% for hTERT) in comparison with the gene expression by the conventional cancer diagnostic method (AFP).

In addition, it was revealed that the cancer diagnostic method using the gene expression (AFP mRNA) according the present invention had higher sensitivity (69.3% for AFP, 71.6% for AFP mRNA) and specificity (60.0% for AFP, 67.5% for AFP mRNA) in comparison with the cancer diagnostic method using the conventional gene expression (AFP).

These results indicate the cancer diagnostic method using the gene expression (hTERT mRNA) according to the invention is effective for the diagnosis of the metastatic malignant tumors in general. Furthermore, the cancer diagnostic method using the gene expression (hTERT mRNA) according to the invention allows screening for the presence of cancer cells from RNA in body fluid at the time of a health check to detect cancer cells in early stage or to detect recurrence of tumor in many patients to significantly improve prognosis.

Finally, the cancer diagnostic method using the gene expression (AFP mRNA) according to the invention allows screening for the presence of cancer cells from RNA in body fluid at the time of a health check to detect cancer cells in early stage or to detect recurrence of tumor in many patients to significantly improve prognosis.

In addition, although the example that RNA was extracted from the blood sample was used as the cancer diagnostic method according to the present invention in the best mode for carrying out the invention of the present specification, it is not limited to a case in which RNA is extracted from blood samples in the cancer diagnostic method according the present invention and RNA may be extracted from the body fluid other than blood samples.

### INDUSTRIAL APPLICABILITY

As described above, the cancer diagnostic method according to the present invention is capable of detecting the evidence indicating the presence of cancer cells in early stage cancer in blood to eradicate the cancer cells in an early stage with medical approaches.

In addition, the presence or absence of cancer cell is accurately detected in comparison with the case that telomerase (hTERT) or AFP is detected from the biopsy of established cancer tissue or metastasized tissue since the sample containing mRNA is obtained from the blood [in the present method].

In addition, by appropriately designing the primers used for the PCR reaction upon running the PCR reaction, the evidence indicating the presence of cancer cells in early stage cancer can be detected in blood.

The cancer diagnostic method according to the present invention is highly valuable in the field of medicine.

What is claimed is:

1. A cancer diagnostic method comprised of;
a process to obtain the sample containing RNA only as a somatic cell and cancer cell fraction from body fluid and
a process having a reverse transcription reaction step to generate cDNA using reverse transcriptase from the sample containing said RNA only and
a PCR reaction step utilizing fluorescent dye using the following primers for hTERT, CGGAAGAGTGTCTGGAGCAA and GGATGAAGCGGAGTCTGGA to quantify said PCR product amplified by said PCR reaction using the fluorescent dye binding to the PCR product.

2. A cancer diagnostic method comprised of;
a process to obtain the sample containing said RNA only as a somatic cell and cancer cell component from body fluid,
a process having a reverse transcription reaction step to generate cDNA using reverse transcriptase from the sample containing RNA and a PCR reaction step utilizing fluorescent dye using the following primers for AFP, CCAGAAACTAGTCCTGGATGT and CGTGGTCAGTTTGCAGCATT to quantify said PCR product amplified by said PCR reaction using the fluorescent dye binding to the PCR product.
